(19) United States
(12) Patent Application Publication
(10) Pub. No.: US 2014/0324090 A1
(43) Pub. Date: Oct. 30, 2014

(54) Compound Needle

(71) Applicants: Ronny Kafiluddi, Loudonville, NY (US); Ersno Eromo, Ghent, NY (US)

(72) Inventors: Ronny Kafiluddi, Loudonville, NY (US); Ersno Eromo, Ghent, NY (US)

(21) Appl. No.: 14/197,666

(22) Filed: Mar. 5, 2014

Related U.S. Application Data
(60) Provisional application No. 61/772,565, filed on Mar. 5, 2013.

(51) Int. Cl. A61B 17/34

(52) U.S. Cl. CPC A61B 17/3417; A61B 2017/3454
USPC 606/186

(57) ABSTRACT

A compound needle comprising an outer needle shaft having an outer needle tip and an inner needle shaft having an inner needle tip wherein one of the needle tip is sharper than the other needle tip. The outer needle tip can be formed of a flexible material and/or have a flexible/movable connection to the outer shaft, which enables the inner needle tip to be deployed and/or retracted through the outer needle tip.

### REFERENCE TO RELATED APPLICATIONS

This application claims the benefit of U.S. Provisional Application No. 61/772,565 filed on Mar. 5, 2013, the entire disclosure of which is hereby incorporated by reference in its entirety.

### TECHNICAL FIELD

The disclosure relates generally to needles, and more particularly, to a compound needle which can be configured with a sharp tip or a blunt tip.

### BACKGROUND ART

Spinal needles frequently are used to perform diagnostic or therapeutic injections in the spine. Currently needles have either a sharp, pencil point or blunt tip. Sharp tipped needles are the most frequently used. An illustrative procedure which utilizes spinal needles includes a selective nerve root or transforaminal injection in order to deposit medicine in close proximity to the spinal nerves.

A problem with the use of a sharp tipped needle is that it may result in nerve damage when it is placed intra-neurally. In addition to nerve damage, vascular injury also can result from a sharp needle piercing a spinal vessel. Furthermore, particulate matter may be accidentally deposited into a spinal artery (which is in close proximity to the target nerve) during a procedure. Such an accidental deposit may result in obstruction of arterial flow to the spine and subsequently in ischemia of the spinal cord which may result in chronic neuropathic pain and even paralysis.

In order to significantly decrease the incidence of neural or vascular damage, blunt needles were developed. A blunt needle tends to displace the nervous structure or blood vessel, rather than puncturing the structure. A blunt pencil needle tip on a spinal needle may still puncture or damage the nerve or artery, but the chance of such damage is less as compared to a sharp needle tip. It has been clinically shown that the incidence of nerve injuries or injuries to vasculature is significantly less when blunt needles are used compared to the use of sharp needles. However, a spinal needle with a blunt needle tip is unable to penetrate the skin and deeper fascial tissue layers.

As a result, current procedures use a sharp needle followed by a blunt needle. In particular, an angiocatheter (including a sharp needle with a plastic cannula surrounding it) is used initially. The angiocatheter with the sharp needle is able to pierce the skin and deeper tissues easily. Once this angiocatheter is placed in proximity to the target nerve, the sharp needle is withdrawn and the plastic cannula left behind. Subsequently, a blunt spinal needle is advanced thru the plastic cannula towards the target nerve until it is placed in close proximity to the nerve.

However, the blunt spinal needle often still needs to be advanced through fascia due to a limited length of the plastic cannula (e.g., typically approximately 1.5 inches), thereby resulting in a “jerky” motion of the blunt spinal needle as it moves toward the nerve. A fluoroscope and contrast material can be used to verify a position of the blunt needle. In particular, the contrast material will spread along the nerve sleeve indicating correct needle placement. Advancing a blunt needle slightly is not likely to puncture a targeted nerve or close-by blood vessel since the tip of the needle is blunt. However, the “jerky” motion can still result in nerve damage since the nerve may be approximated with a high velocity.

### SUMMARY

The disclosure relates generally to needles, and more particularly, to a compound needle which can be configured with a sharp tip or a blunt tip.

Some aspects provide a compound needle including a needle with an outer shaft and a sharp needle tip (“sharp needle”) and a needle with a blunt needle tip (“blunt needle”) located inside the shaft of the sharp needle. The sharp needle tip can be formed of a flexible material and/or have a flexible/movable connection to the outer shaft, which enables the blunt needle to be deployed and/or retracted through the sharp needle tip.

One aspect provides a compound needle including one or more features as described herein. Other aspects provide methods, products, systems, and methods of using and generating each, which include a compound needle described herein.

The illustrative aspects are designed to solve one or more of the problems herein described and/or one or more other problems not discussed.

### BRIEF DESCRIPTION OF THE DRAWINGS

These and other features of the disclosure will be more readily understood from the following detailed description of the various aspects according to the present disclosure taken in conjunction with the accompanying drawings that depict various aspects according to the present disclosure.

FIG. 1A is a side view of an example of an inner needle with a distal needle tip that has a blunt end with a rounded edge.

FIG. 1B is a side view of an example of an outer needle with a distal needle tip that has a sharp end.

FIG. 2A is a side view of an example of a compound needle where the inner needle is housed inside the hollow shaft of the outer needle.

FIG. 2B is a front view of the distal needle tip of the outer needle of the compound needle in FIG. 2A.

FIG. 2C is a side view of an example of a compound needle where the inner needle is deployed and extends out from the distal needle tip of the outer needle.

FIG. 2D is a front view of the distal needle tip of both the outer needle and the inner needle of the compound needle in FIG. 2C.

FIG. 3A is a side view of an example of a curved inner needle with a distal needle tip that has a blunt end with a rounded edge.

FIG. 3B is a side view of an example of a curved outer needle with a distal needle tip that has a sharp end.

FIG. 4A is a side view of an example of a curved compound needle where the inner needle is housed inside the hollow shaft of the outer needle.

FIG. 4B is a side view of an example of a curved compound needle where the inner needle is deployed and extends out from the distal needle tip of the outer needle.

FIG. 5A is a side view of an example of an inner needle with helical ridges on the proximal end of the inner needle shaft.

FIG. 5B is a side view of an example of an outer needle with a rotatable member.

It is noted that the drawings may not be to scale. The drawings are intended to depict only typical aspects, and therefore should not be considered as limiting the scope of the invention. In the drawings, like numbering represents like elements between the drawings.

### DETAILED DESCRIPTION

As indicated above, aspects provide a compound needle including a needle with an outer shaft and a sharp needle tip (“sharp needle”) and a needle with a blunt needle tip (“blunt needle”) located inside the shaft of the sharp needle. The blunt needle can serve as a stylet inside the outer sharp needle. The sharp needle tip can be formed of a flexible material and/or have a flexible/movable connection, which enables the blunt needle to be deployed and/or retracted through the sharp needle tip. For example, the blunt needle can be deployed by pushing a hub of the blunt needle so that the blunt needle tip end pushes through the flexible tip of the sharp needle. The deployed blunt needle can be locked into position, e.g., using a spring loaded ball, which extends through an opening in the outer shaft of the sharp needle tip. As used herein, unless otherwise noted, the term “set” means one or more (i.e., at least one) and the phrase “any solution” means any now known or later developed solution.

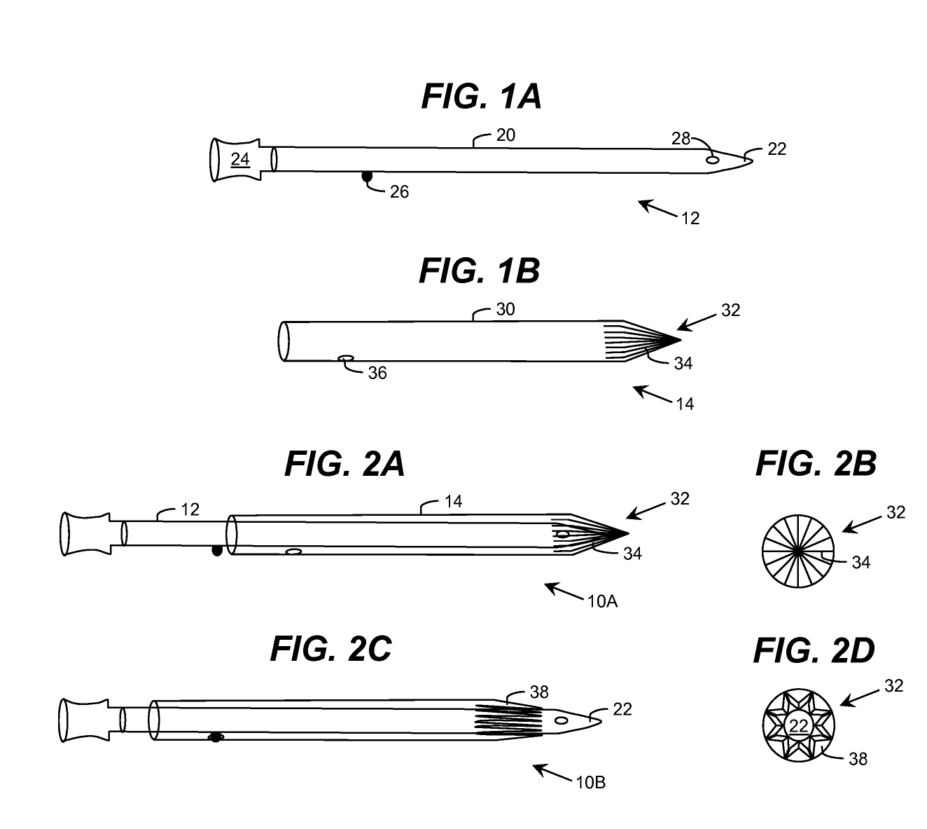

Turning to the drawings, FIGS. 1A and 1B show an illustrative blunt needle 12 and an illustrative sharp needle 14 according to embodiments. The blunt needle 12 can include a shaft 20 having a first end with a blunt needle tip 22 and an opposing end with a hub 24. The sharp needle 14 can include a hollow shaft 30, which terminates at a sharp needle tip 32. In an embodiment, the sharp needle tip 32 includes a plurality of perforating blades 34. The perforating blades 34 can be formed using any solution. For example, a blade 34 can be formed by a cut into the material of the tip 32, formed and attached (e.g., soldered) to the tip 32, and/or the like.

The blunt needle 12 is sized to be located within an interior of the sharp needle 14 to form a compound needle, which can be configured as a blunt needle or a sharp needle. To this extent, FIGS. 2A and 2B show side and front views of an illustrative compound needle in a sharp needle configuration 10A, while FIGS. 2C and 2D show side and front views the illustrative compound needle in a blunt needle configuration 10B according to embodiments. In FIGS. 2A and 2B, the blunt needle 12 is located entirely within the sharp needle 14, and the sharp needle tip 32 forms a closed, pencil like sharpened point on one end of the compound needle. In this configuration, the compound needle can be used to readily pierce tissue using the sharp needle tip 32.

As described herein, the sharp needle tip 32 is configured to allow the blunt needle tip 22 to be deployed there through. As illustrated in FIGS. 2C and 2D, in order to allow the blunt needle tip 22 to deploy, the sharp needle tip 32 can be formed of a plurality of flexible members 38, which can separate as the blunt needle tip 22 is pushed against an interior of the sharp needle tip 32. Furthermore, the flexible members 38 can be configured to return to their original positions when the blunt needle tip 22 is retracted. For example, each flexible member 38 can comprise a corresponding perforating blade 34, which is attached to the sharp needle shaft 30 using a flexible material and/or a flexible connection. The flexible material/connection can comprise any type of material/connector having a sufficient stiffness to enable the sharp needle tip 32 to be utilized to readily pierce tissue, but a sufficient flexibility to allow the blunt needle tip 22 to be deployed through the sharp needle tip 32. In an embodiment, each needle 12, 14 is formed of a material, such as stainless steel or another alloy (e.g., tin), having sufficient strength to resist breaking off while in use (e.g., in tissue).

In an embodiment, the blunt needle 12 can be configured to be locked into a position in the deployed and/or non-deployed positions. To this extent, the blunt needle shaft 20 is shown including a spring loaded ball 26 (FIG. 1A), while the sharp needle shaft 30 is shown including an opening 36 (FIG. 1B). As shown in FIGS. 2A and 2C, when the blunt needle tip 22 is deployed through the sharp needle tip 32, the spring loaded ball 26 can align with the opening 36, thereby locking the blunt needle tip 22 in place. It is understood that the blunt needle 12 and the hollow interior of the sharp needle shaft 30 can be configured to ensure that the spring loaded ball 26 and the opening 36 will align (e.g., by having complementary shapes that only allow the blunt needle 12 to be inserted such that the alignment will occur). Furthermore, it is understood that while only a single opening 36 is shown, the sharp needle shaft 30 can include a plurality of openings, which can correspond to a set of non-deployed positions for the blunt needle 12 and a set of deployed positions for the blunt needle 12.

In an embodiment, the compound needle can be used to perform a treatment procedure on a target nerve. In an illustrative application, the blunt needle 12 can be used to inject a local anesthetic to anesthetize the target nerve. In another illustrative application, the blunt needle 12 can be used to ablate the target nerve. In either case, the shaft 20 of the blunt needle 12 can be hollow to enable delivery of a treatment to the target nerve through a port 28 (FIG. 1A) located adjacent to the blunt needle tip 22. For example, the local anesthetic can be injected through the hub 24 of the blunt needle 12 and exit the port 28 in a location adjacent to the target nerve. Similarly, a heating probe of a radiofrequency generator can be placed into the blunt needle 12 though the hub 24 and located adjacent to the port 28 to ablate the target nerve. Regardless, the blunt needle 12 and the sharp needle 14 can be configured to enable a sufficient length of the blunt needle 12 to be deployed beyond the sharp needle tip 32 so that the port 28 is also beyond the sharp needle tip 32.

A pencil tipped needle, such as that shown in FIGS. 1A-2D, is not readily steerable. In a further embodiment, compound needles having any one of a plurality of needle tip configurations can be provided. For example, a first needle tip configuration can comprise a straight tip needle, such as that shown and described in conjunction with FIGS. 1A-2D. A second needle tip configuration can comprise a curved tip needle, which can enable an operator to better steer the compound needle towards a targeted nerve. In a still further embodiment, the compound needle can include a combination straight/curved tip needle, where one needle tip (sharp or blunt) is straight or curved, while the other needle tip is the other of straight or curved.

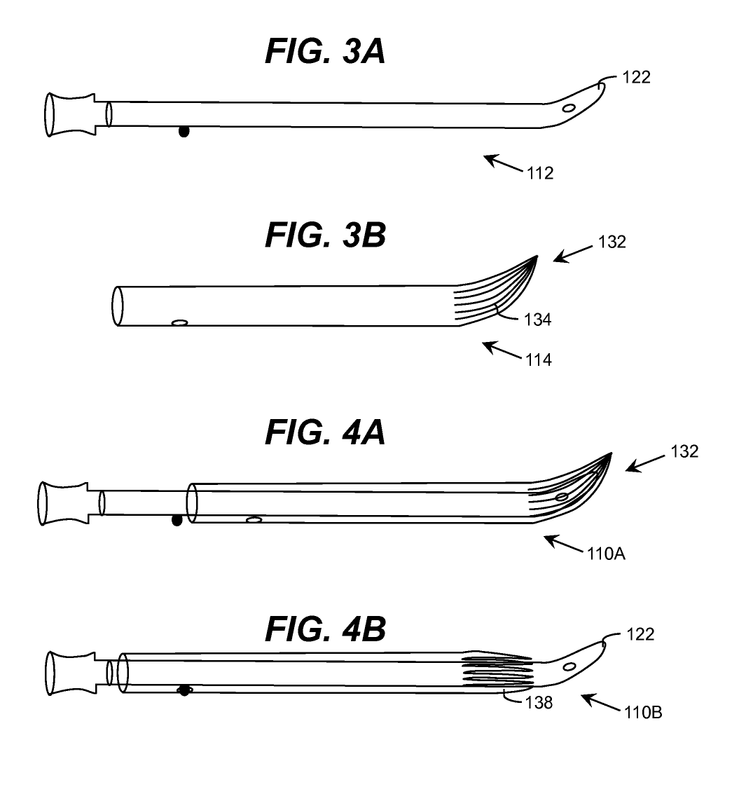

FIGS. 3A and 3B show an illustrative blunt needle 112 and an illustrative sharp needle 114 according to embodiments. Additionally, FIGS. 4A and 4B show an illustrative compound needle formed using the blunt needle 112 and the sharp needle 114 in a sharp needle configuration 110A and a blunt needle configuration 110B according to embodiments. The blunt needle 112 and the sharp needle 114 can include various components and features similar to those described herein in conjunction with FIGS. 1A-2D. As a result, these features and components are not further described for clarity. However, the blunt needle 112 includes a curved blunt needle tip 122 and the sharp needle 114 includes a curved sharp needle tip 132 comprising a plurality of curved perforating blades 134 and a plurality of curved flexible members 138. Use of a curved needle tip 122, 132 can enable an operator to better steer the needle tip 122, 132 towards a targeted nerve. As illustrated, the curved flexible members 138 of the needle tip 122 can be configured to flatten when the blunt needle 112 is deployed there through such that the curved perforating blades 134 are substantially snug to the shaft of the blunt needle 112. In this manner, the curved perforating blades 134 are less likely to damage any tissue surrounding the compound needle. For example, the plurality of flexible members 138 can be configured such that they cannot separate by more than a small amount. In this case, when the shaft of the blunt needle forces the lower flexible members 138 lower, they will pull the upper flexible members 138 down to the shaft of the blunt needle.

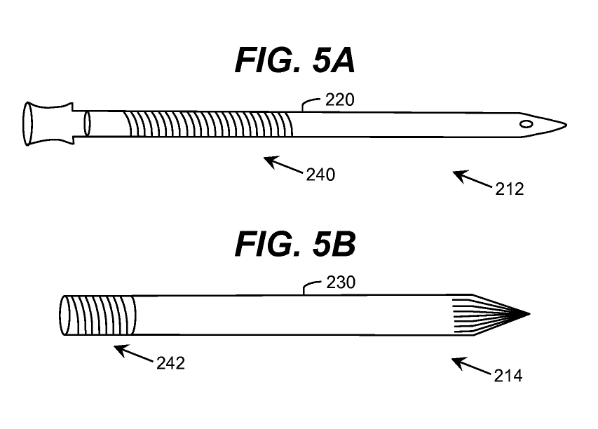

As discussed herein, the blunt needle can be deployed through the tip of the sharp needle by pushing on a hub of the blunt needle. However, it is understood that an embodiment of the compound needle can enable finer control of the deployment of the blunt needle and/or continual locking of the blunt needle in a position. For example, FIGS. 5A and 5B show an illustrative blunt needle 212 and an illustrative sharp needle 214 according to embodiments. As previously described, the blunt needle 212 can be configured to be located within a hollow shaft 230 of the sharp needle 214. However, in this case, at least a portion of the outer surface of the shaft 220 of the blunt needle 212 is threaded 240. Additionally, the hollow shaft 230 of the sharp needle 214 can include a rotatable member 242 attached to a distal end thereof. The rotatable member 242 can be attached in a manner that enables an operator to rotate the rotatable member 242 without rotating the shaft 230. The rotatable member 242 can have an interior surface with threading complementary to the threaded portion 240 of the exterior of the blunt needle shaft 220. In this case, an operator (human or computer) can rotate the rotatable member 242 to deploy and/or retract the blunt needle 212 as described herein. Furthermore, the complementary threading can secure the blunt needle 212 in any of various positions. It is understood that the threaded member and threading described herein is only illustrative of various configurations, which can be utilized to enable finer, continual adjustment of the location of the blunt needle 212 with respect to the sharp needle 214.

Aspects of the needle described herein can enable various procedures, which currently use two different needles and a plastic cannula, to be performed using a single needle, thereby saving time (e.g., one needle entry), expense (e.g., no need for an angiocatheter), and/or the like. A needle described herein can be manufactured in any of various sizes and configurations. For example, a length of the outer needle can vary between approximately 3.5 and approximately 6 inches, and an inner diameter of the inner needle can vary between approximately 18 gauge and approximately 25 gauge (e.g., 18G, 20G, 22G, 25G, and/or the like). Regardless, prior to distribution, a needle described herein can be sterilized using any solution and packaged in a sterilized pouch.

A needle described herein can be used in various environments where safe access to a nerve, such as a spinal nerve, a peripheral nerve (e.g., the nerves of the brachial plexus, sciatic nerve, pudental, etc.), and/or the like, is needed. Such environments can include clinical settings such as a medical office, an ambulatory surgery center, a hospital, and/or the like, in which a spinal injection may be performed for diagnostic and/or therapeutic purposes. Specialists who may use the needle include: orthopedic surgeons, neurosurgeons, radiologists, physical medicine and rehabilitation specialists, pain management physicians, and/or the like.

During use of the compound needle, the sharp needle can be used to readily pierce tissue and can be located in close proximity to a targeted nerve/blood vessel using, for example, a fluoroscope, ultrasound, and/or the like. Once the sharp needle is sufficiently close to the targeted nerve/blood vessel but sufficiently far away so as not to puncture the targeted nerve/blood vessel, the blunt needle can be deployed, e.g., by pushing a hub of the blunt needle to extend the blunt needle tip thru the flexible tip of the sharp needle. Once deployed, the blunt needle can be locked into position and steered/advanced, e.g., fluoroscopically with the use of contrast, to a location adjacent to the targeted nerve/blood vessel. Since there usually is no fascial layer around a nerve, the blunt needle can be placed adjacent to the target nerve without a jerky motion, which could damage the target nerve.

In an embodiment, the compound needle can be used to anesthetize or ablate a nerve, such as a peripheral nerve. To this extent, the compound needle can be included as a component of a system for anesthetizing and/or ablating a nerve. For example, using such a system, a local anesthetic can be injected through the blunt needle to anesthetize the nerve, a heating probe from a radiofrequency generator can be placed within the shaft of the blunt needle to ablate the nerve, and/or the like.

The foregoing description of various aspects has been presented for purposes of illustration and description. It is not intended to be exhaustive or limited to the precise form disclosed, and obviously, many modifications and variations are possible. Such modifications and variations that may be apparent to an individual in the art are included within the scope of the invention as defined by the accompanying claims.

1-2. (canceled)

3. A compound needle comprising:
an inner shaft having a first needle tip; and
an outer shaft having a second needle tip, wherein one of the first and second needle tips is sharper than the other of the first and second needle tips.

4. The compound needle of claim 3, wherein the second needle tip is formed from a flexible material.

5. The compound needle of claim 3, wherein the first needle tip is sharper than the second needle tip.

6. The compound needle of claim 3, wherein the second needle tip comprises perforating blades.

7. The compound needle of claim 3, the inner shaft is deployable and is disposed within a hollow core of the outer shaft, and wherein the inner shaft, when deployed, causes the first needle tip to:
extend through the second needle tip; and
be locked in a position when fully extended.

8. The compound needle of claim 7, wherein the first needle tip, when locked into position, uses a spring loaded ball that extends through an opening in the outer shaft.

9. The compound needle of claim 3, wherein the first needle tip and the second needle tip is curved.

10. The compound needle of claim 3, wherein the inner shaft is threaded and the outer shaft comprise a rotatable member.
